(19) United States
(12) Patent Application Publication
(10) Pub. No.: US 2022/0092492 A1
(43) Pub. Date: Mar. 24, 2022

(54) TEMPORAL AND SPATIAL SUPPLY CHAIN RISK ANALYSIS

(71) Applicant: International Business Machines Corporation, Armonk, NY (US)

(72) Inventors: Zachary A. Silverstein, Jacksonville, FL (US); Juan C. Lopez, Tampa, FL (US)

(21) Appl. No.: 17/026,745

(22) Filed: Sep. 21, 2020

(51) Int. Cl. G06Q 10/06; G06Q 50/28; G16H 50/80; G06N 3/08

(52) U.S. Cl. CPC G06Q 10/0635; G06Q 50/28; G06N 3/08; G06Q 10/067; G16H 50/80; G06Q 10/06375

(57) ABSTRACT

Data of a supply chain is received. The data includes components moving through a locations at points in time. Risks are identified that relate to at least some of the locations. Likelihoods of each of the risks impacting any of the components at any of the locations at any of the points of time are calculated by simulating the risks spreading across space and time. A risk of the risks is identified as having more than a threshold likelihood of impacting at least one component at a respective location at a respective point in time. A change to the supply chain that lowers a likelihood of the risk impacting the at least one component at the respective location at the respective point in time to below the threshold likelihood is provided to a user.

### BACKGROUND

Supply chain management is a robust industry that involves organizing components from supply to manufacture to sale. For example, a conventional supply chain management software solution may assist a user in organizing components into a plurality of individual logistical routes from one or more supplies into one or more manufacturing, assembling, and/or preparation facilities, and therein organizing logistical routes for finished versions of these components to be transported to one or more destinations where these components/products may be sold and/or delivered for use. Put differently, conventional supply chain management software solutions may help users organize how components are coming from various sources, and therein organize how the finished subsequent products are then transported away. For example, a user may store numerous options for suppliers, numerous options for manufacturing plants, and/or numerous options for transportation within a conventional supply chain management solution to better compare different prices and/or qualities of a final product depending upon how a supply chain is constructed.

### SUMMARY

Aspects of the present disclosure relate to a method, system, and computer program product relating to a temporal and spatial supply chain risk analysis. For example, a method includes receiving data of a supply chain. The data includes a plurality of components each moving through a plurality of locations at a plurality of points in time. The method also includes identifying a plurality of risks that relate to at least some of the plurality of locations. The method also includes calculating likelihoods of each of the plurality of risks impacting any of the plurality of components at any of the plurality of locations at any of the plurality of points of time by simulating the risks spreading across space and time. The method also includes identifying that a risk of the plurality of risks has more than a threshold likelihood of impacting at least one component of the plurality of components at a respective location of the plurality of locations at a future point in time of the plurality of points in time. The method also includes providing, to a user, a change to the supply chain that lowers a likelihood of the risk impacting the at least one component at the respective location at the respective point in time to below the threshold likelihood. A system and computer product configured to perform the above method are also disclosed.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a conceptual diagram of one embodiment of a computing environment in which a controller identifies potential risks to a supply chain and simulates how these risks may change over time and space to impact the supply chain.

FIG. 2 depicts a conceptual diagram of a supply chain and other options for a supply chain that may be evaluated by the controller of FIG. 1 regarding one or more risks in simulating how the one or more risks may impact the supply chain.

FIG. 3 depicts a conceptual box diagram of one embodiment of the controller of FIG. 1.

FIG. 4 depicts a flowchart of one embodiment of an example method of a controller evaluating risks to a supply chain by simulating how the risks change regarding the supply chain over time and space.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to managing supply chains, while more particular aspects of the present disclosure relate to simulating the interrelation of various risks over time and space in comparison to how components of a supply chain are managed (e.g., gathered/transported/assembled/delivered) over time and space. While the present disclosure is not necessarily limited to such applications, various aspects of the disclosure may be appreciated through a discussion of various examples using this context.

Many modern products require complex supply chains to fully realize these products. For example, a modern product such as a cell phone may include an initial supply chain leg of raw material extraction, a second supply chain leg of component manufacture, a third supply chain leg of assembly, and/or a fourth supply chain leg of product delivery. Conventional supply chain management solutions may be configured to assist a user in constructing these supply chains. For example, conventional supply chain management solutions may provide ways in which a user may organize and visualize supply chains. A single supply chain may include components and assembly across numerous continents using hundreds of transportation vehicles to dozens of locations for multi-step production. Further, an unanticipated delay at one point in the supply chain may impact the entirety of the supply chain. For example, if a supply chain requires a rare-earth element as mined from a set of mines as per a contract to assemble a component that is then assembled into a final product, and a natural disaster disrupts production at that set of mines, the entire supply chain may grind to a halt as a result of that single failure of that single component.

As such, conventional supply chain management systems and methods are set up to quickly enable a user to change supply chains in response to a detected current event, such that an amount of inevitable downtime may be minimized. However, by the time an event is detected it may be too late to avoid a slowdown. As such, some human users may pre-emptively monitor events to determine when a risk might occur that will impact a supply chain, such that the supply chain should be changed.

However, it may be difficult for a human mind to understand how events interrelate as they spread out in time and space, such that it may be difficult and/or impossible for a user to accurately predict when a risk has a high likelihood of impacting the supply chain and also determine the route that has the smallest disruption while still staying under a risk threshold. Put differently, it may be difficult or impossible for a human to mentally comprehend how a supply chain that has hundreds of events that spread out in time and space that interrelate can be impacted by a risk that itself also moves out through time and space, much less determine which of the hundreds of alternative options for mitigating this risk best works within the rest of the structure of how the supply chain is currently operating in space and time. Further, if a user is too risk-averse and decides to change a supply chain where a risk was not likely to materialize and/or not likely to impact the supply chain, the user may risk introducing an inefficiency into the system via this change. For example, even if a user correctly identifies that a risk event is going to happen at a location that relates to the supply chain, the user may fail to determine that the risk is going to exist at this location at a point in time at which the supply chain does not utilize that location, such that modifying the supply chain per this risk would needlessly disrupt the supply chain. Similarly, even if a user correctly identifies that a risk event is going to happen at a location that relates to the supply chain at a time that components of the supply chain are moving through that location, the user may introduce added inefficiency by moving this step of the supply chain to a different continent, failing to identify that an adjacent location on the same continent is able to move components through with an acceptable low rate of impact.

Aspects of this disclosure may improve an ability to accurately predict when risks that change, grow, and/or move through space and time have a sufficiently high likelihood of impacting components at one or more specific points in time of a supply chain, and therein determine one or more options that are below the risk threshold while minimizing an amount of change and/or inefficiency (e.g., inefficiency between the original supply chain and the changed supply chain, where inefficiency relates to extra time and/or money that it takes for the supply chain to successfully conclude). For example, aspects of this disclosure may identify potential risks and then calculate a likelihood of these risks impacting the supply chain by simulating these risks across time and space to identify intersections between the risk propagation and the simulated supply chain through time and space.

One or more computing controllers that include one or more processors executing instructions stored on one or more computing memories (these computing controllers discussed generically herein as being a single computing controller) may simulate these risks across time and space to determine if any will overlap with expected components at expected locations at expected points in time of the supply chain. If the controller determines that a risk of such overlap is greater than a threshold such that the risk has more than a threshold likelihood of impacting the supply chain (e.g., where impacting the supply chain means causing a component to fail to successfully traverse across a predetermined location within a predetermined time window at a predetermined point in time), the controller may alert a user. In some examples, the controller may further identify one or more options that would maintain the supply chain with a minimized amount of inefficiency while avoiding the risk. The controller may provide these options to a user. In certain examples, the controller may further autonomously execute one or more of these options in response to determining that the change to the supply chain satisfied one or more criteria (e.g., that it can be completed with a high confidence score, that a likelihood of impact is relatively high, and/or that a timing of the risk is relatively soon).

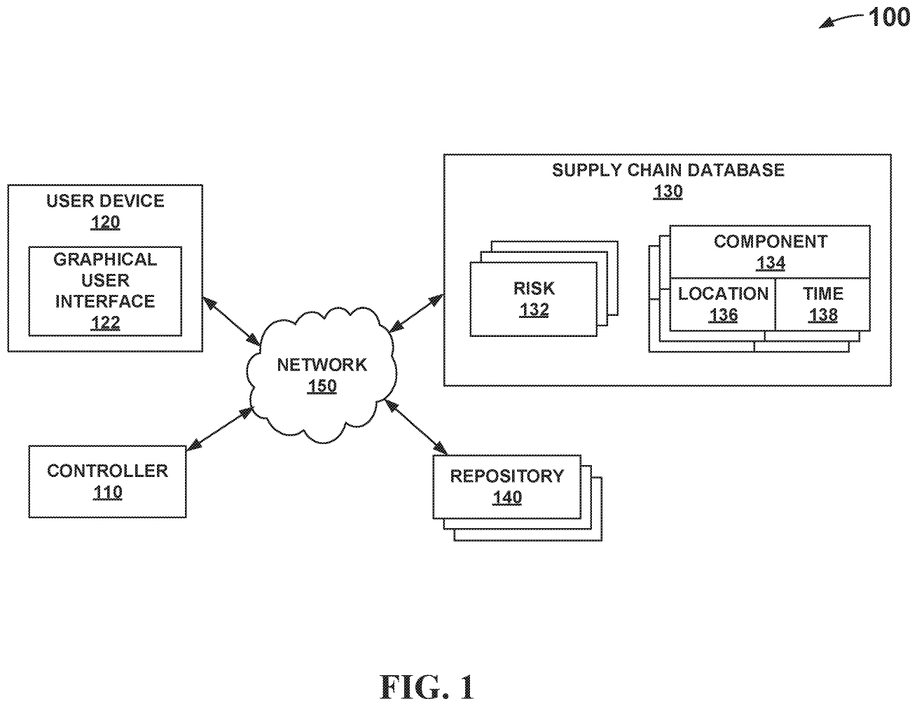

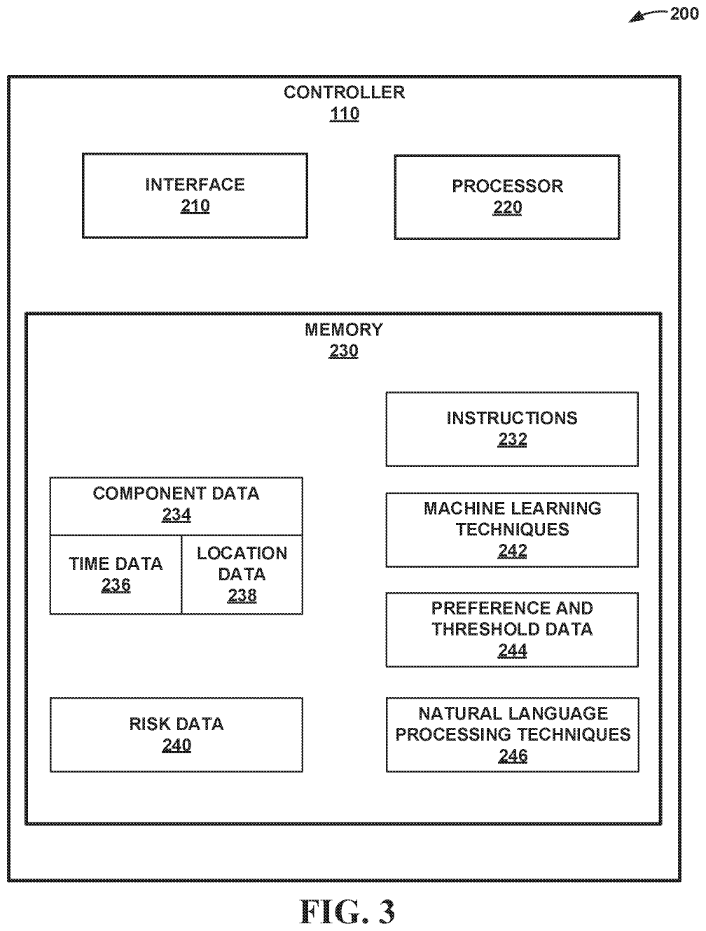

For example, FIG. 1 depicts environment 100 that includes controller 110 that is configured to calculate if identified risks are likely to impact a supply chain by simulating the risks as they propagate through space and time while simulating the supply chain operating through space and time. Controller 110 may include a computing device, such as computing device 200 of FIG. 3 that includes a processor communicatively coupled to a memory that includes instructions that, when executed by the processor, cause controller 110 to execute the operations described below.

Controller 110 may analyze a supply chain in response to a request and/or prompt from a user. For example, a user may send a prompt via user device 120, which may be a computing device (e.g., a computing device similar to computing device 200 of FIG. 3). In response to receiving such a prompt, controller 110 may gather and/or receive information on the supply chain. In some examples, controller 110 may receive an initial set of information (and/or a complete set of information) on the supply chain within this initial prompt from the user. Alternatively, or additionally, controller 110 may receive and/or gather this information from supply chain database 130.

Supply chain database 130 may include one or more computing devices (e.g., computing devices similar to the computing device 200 of FIG. 3) that are owned by an organization that handles the supply chain (e.g., the organization that sells the product which supply chain manufactures and/or delivers). Though controller 110 is depicted as part of a separate and discrete device in relation to supply chain database 130, in some examples controller 110 may be integrated into supply chain database 130.

Supply chain database 130 may include data on one or more supply chains of an organization. As used herein, a supply chain includes a logistical and/or organizational data structure that defines the process of components moving through space and time, including all steps from receiving components from a source to delivering finished products to a buyer, where these components are often changed and/or combined into a greater assembly via the supply chain. For example, supply chain database 130 may include data on components 134, which may include objects that physically occupy predetermined locations 136 as they move through the supply chain during predetermined points in time 138. The supply chain, as stored within supply chain database 130, may be gauged to be successful based on the supply chain moving a plurality of components 134 through different predetermined locations 136 at different predetermined points in time 138 to arrive together and/or separately at predetermined locations 136 at predetermined points in time 138. For example, as would be understood by one of ordinary skill in the art, modern supply chains are often finalized with a contractual agreements between supply, transportation, manufacture, and buyer, such that a specific relative timing of where components 134 are at various locations 136 and times 138 may impact performance metrics specified by these contractual agreements. As such, data on components 134, locations 136, and points in time 138 as stored within supply chain database 130 and managed and/or utilized by controller 110 may be structurally connected, such as via data on components 134 and locations 136 and time 138 all being stored within a self-referential two-dimensional or three-dimensional spreadsheet, or the like.

Once controller 110 gathers a sufficient amount of data on the supply chain such that controller 110 has fully defined the relationships between how components 134 need to move locations 136 and points in time 138 in order for the supply chain to be successful, controller 110 may identify one or more risks 132 to the supply chain. As described herein, risks 132 include events and/or occurrences that make it relatively less likely for any component 134 to be able to traverse to and/or through a predetermined location 136 during a predetermined time window at a predetermined point in time 138 as defined by supply chain database 130.

In some examples, controller 110 may receive information regarding risks 132 from user device 120. For example, controller 110 may receive a message from user device 120 that identifies and provides information on risks 132 which controller 110 is to analyze. Upon receiving this information, as depicted in FIG. 1, controller 110 may save this information within supply chain database 130. Additionally, or alternatively, controller 110 identify data on risks 132 as already stored within supply chain database 130.

In some examples, controller 110 may gather data on risks 132 by crawling across one or more repositories 140. For example, controller 110 may receive a request from user device 120 that identifies a supply chain to analyze, after which controller 110 may gather (and/or receive) data on locations 136 and/or points in time 138 of supply chain. Once controller 110 receives data on these locations 136 and/or points in time 138, controller 110 may search across repositories 140 for indications of risks 132 related to these locations 136. For example, controller 110 may search repositories 140 for weather type risks 132 (e.g., hurricanes, wildfires, etc.), viral pandemics risks 132, geopolitical type risks 132, or the like. Controller 110 may use natural language processing (NLP) techniques as known in the art and discussed herein to discover and/or ingest this data once discovered. In some examples, controller 110 may gather data on risks 132 after simulating the supply chain to identify respective points in time 138 at which components 134 will be at locations 136 prior to search through repositories 140 for risks 132 that relate to these locations 136 and/or points in time 138.

Controller 110 may access such repositories 140 and corpus 120 over network 150. Network 150 may include a computing network over which computing messages and/or computing data may be sent and/or received. For example, network 150 may include the Internet, a local area network (LAN), a wide area network (WAN), a wireless network such as a wireless LAN (WLAN), or the like. Network 150 may comprise copper transmission cables, optical transmission fibers, wireless transmission, routers, firewalls, switches, gateway computers and/or edge servers. A network adapter card or network interface in each computing/processing device (e.g., controller 110, user device 120, supply chain database 130, and/or repositories 140) may receive messages and/or instructions from and/or through network 150 and forward the messages and/or instructions for storage or execution or the like to a respective memory or processor of the respective computing/processing device.

Though network 150 is depicted as a single entity in FIG. 1 for purposes of illustration, in other examples network 150 may include a plurality of private or public networks. For example, controller 110, user device 120, and supply chain database 130 may communicate together over a private LAN of network 150 (e.g., as these three may be owned and/or used by the organization that sells a product of the supply chain). Additionally, controller 110 may gather data related to users from repositories 140 over a public portion of network 150 using the Internet.

Once controller 110 has identified (and gathered relevant data on) risks 132, controller 110 may simulate risks 132 through space and time to determine any overlaps and/or interrelations between how risks 132 propagate through space and time and how components 134 are at locations 136 at points in time 138. Controller 110 may calculate specific likelihoods of risks 132 impacting any given component 134 traversing through any given location 136 at any given point in time 138. If controller 110 determines that a likelihood is above a predetermined threshold, controller 110 may provide an alert to a user. For example, controller 110 may provide a graphical indication via graphical user interface of user device 120.

Controller 110 may also identify potential changes to the supply chain that mitigate risks 132. As used herein, a change mitigating a risk may include the change lowering the calculated likelihood of impact to the supply chain below a threshold amount as calculated via simulations of risks 132 and different combinations of components 134 at different locations 136 at different points in time 138. For example, controller 110 may identify alternative locations 136 through which component 134 may traverse. Controller 110 may identify these alternative locations 136 as previously used by other supply chains, previously used by historical iterations of the current supply chain, as current alternatives listed within supply chain database 130, or the like. Alternatively, or additionally, controller 110 may identify alternate suppliers for one or more components 134. In some examples, controller 110 may identify alternative points in time 138 at which the same component 134 may move through the same location 134, perhaps simultaneously moving other components 134 to an “earlier” point in time 138 so that a final full timeline of supply chain is also achieved. Other examples of how controller 110 may change supply chain that are consistent with this disclosure are also possible. Once identified, controller 110 may provide a graphical indication of one or more of these changes to a user via graphical user interface 122 provided on user device 120. In some examples, controller 110 may provide this graphical indication such that a likelihood of risk 132 of the original supply chain is displayed, a likelihood of risk 132 of the provided options is displayed, and/or an amount of determined inefficiency of the options is displayed in the graphical indication.

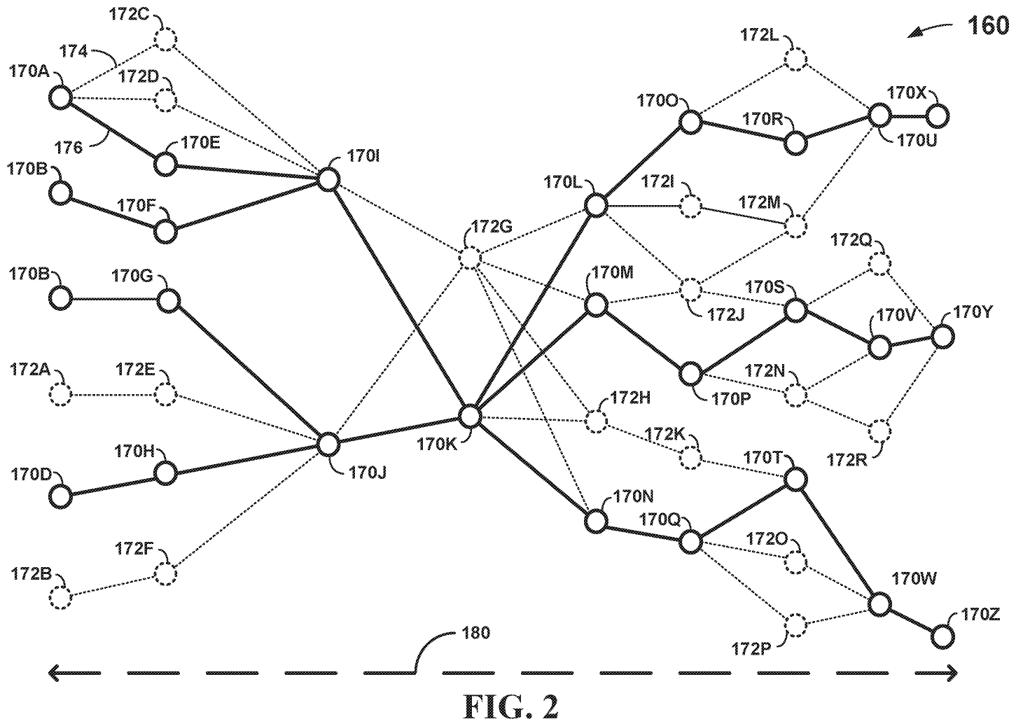

For example, FIG. 2 depicts a conceptual diagram of supply chain 160 with active nodes 170A-170Z (collectively, “active nodes 170”) indicating current paths connected by edges 176 across time and space that components 134 are currently planned to travel according to supply chain 160. Supply chain 160 also includes different optional nodes 172A-172R (collectively, “optional nodes 172”) as connected by edges 174 that depict alternative paths across time and space that components 134 could alternatively take. Supply chain 160 is further depicting as going across time axis 180 that defines a relative spacing between all active nodes 170 and optional nodes 172 in order to successfully execute supply chain 160. For example, time axis 180 of supply chain 160 may not define actual dates, but rather a relative amount of hours, days, weeks, etc. that are allowed to expire between components 134 being at different locations 136.

It is to be understood that the relative number and arrangement of active nodes 170 and optional nodes 172 within FIG. 2 is provided for purposes of illustration only. Further, for the sake of illustration, there are less optional nodes 172 than active nodes 170, though in other examples there may more optional nodes 172 than active nodes 170, such as an order of magnitude more optional nodes 172 than active nodes 170. For example, as would be understood by one of ordinary skill in the art, in reality a supply chain may include hundreds or thousands of different active nodes 170 (indicating currently planned moments in time and space that all respective components 134 must be at in order to have the supply chain succeed), with thousands or tens of thousands optional nodes 172 that are identified and analyzed by controller 110. As such, as would be understood by one of ordinary skill in the art, it can be functionally impossible for a user to reliably understand all of the ways in which risks 132 may evolve to impact a supply chain, much less identify the best ways to change the supply chain to mitigate these impacts. As such, configuring controller 110 to not only accurately identify when risks 132 have more than a threshold likelihood of impacting the supply chain but also simulating how the propagating risks 132 impact each of the optional nodes 172 to determine the path that is under the threshold amount of risk while also having the least amount of inefficiency may greatly improve an ability to manage risks 132 to supply chain 160.

Supply chain 160 may include four components 134 starting at four active nodes 170A-170D, before these components 134 are first combined into two sub-assembly components 134 at active nodes 170I, 170J, then combined into a single product component 134 at active node 170K, and then shipped to three different retail outlets at active nodes 170X, 170Y, 170Z. However, as depicted, controller 110 may identify a plurality of optional nodes 172 that indicate different suppliers, different assembly plants, different transporters, different transportation routes, or the like. In some examples, controller 110 may access a predetermined record of all allowed/possible alternative options of optional nodes 172 (e.g., a record as maintained by organization of the supply chain), whereas in other examples controller 110 may curate alternate options by crawling through repositories 140 and/or supply chain database 130 (e.g., where supply chain database 130 includes other current and/or historical supply chains of a user).

Controller 110 may determine that, e.g., risks 132 of a pandemic viral outbreak is starting in one portion of the world while a hurricane is forming in another part of the world. It may be difficult or impossible for a user to accurately visualize how these two may grow over time to impact various locations 136 of a supply chain. As such, controller 110 may gather data on both the viral pandemic outbreak risk and the hurricane risk, and execute simulations regarding how these risks 132 may impact supply chain 160. Controller 110 may execute a predetermined number of simulations (e.g., 1,000 simulations, or 10,000 simulations) to determine a baseline of outcomes. Controller 110 may further use machine learning models such as neural networks to compare current data regarding the identified risks 132 against historical data regarding similar risks 132 to execute these simulations and identify likelihoods of impact (and/or identifying potential inefficiencies of various options).

For example, via these simulations, controller 110 may calculate that there is a 5% likelihood of the viral pandemic impacting transportation of first component 134 through location 136 related to active node 170E, a 35% likelihood of the viral pandemic impacting a supply of fourth component 134 related to active node 170D, and a 25% likelihood of the hurricane impacting a final assembly at active node 170K. Controller 110 may identify that a risk threshold of the respective user and/or supply chain is 10%. Accordingly, controller 110 may identify that risk 132 related to node 170E may be ignored. Further, controller 110 may identify that risk 132 relating to node 170D exceeds the risk threshold and therein identify optional nodes 172A and 172B as potential changes to supply chain. Controller 110 may identify respective risks 132 and/or inefficiencies (if any) of optional nodes 172A, 172B. Controller 110 may further identify that risk 132 associated with node 170K exceeds the threshold and therein identify optional node 172G. Controller 110 may determine whether or not any of these optional nodes 172A, 172B, 172G satisfy predetermined criteria or requisites for autonomous action. For example, controller 110 may determine that risks 132 related to transportation-related active nodes 170D may satisfy a set of criteria that relates to sending an immediate message to the transporting agent to avoid the viral pandemic.

As described above, controller 110 may include a computing device with a processor configured to execute instructions stored on a memory to execute the techniques described herein. For example, FIG. 3 is a conceptual box diagram of such computing device 200 of controller 110. While computing device 200 is depicted as a single entity (e.g., within a single housing) for the purposes of illustration, in other examples, controller 110 may be stored within computing device 200 that includes two or more discrete physical systems (e.g., within two or more discrete housings). Computing device 200 may include interfaces 210, processor 220, and memory 230. Computing device 200 may include any number or amount of interface(s) 210, processor(s) 220, and/or memory(s) 230.

Computing device 200 may include components that enable controller 110 to communicate with (e.g., send data to and receive and utilize data transmitted by) devices that are external to controller 110. For example, controller 110 may include interface 210 that is configured to enable controller 110 and components within controller 110 (e.g., such as processor 220) to communicate with entities external to controller 110. Specifically, interface 210 may be configured to enable components of controller 110 to communicate with user device 120, supply chain database 130, repositories 140, or the like. Interface 210 may include one or more network interface cards, such as Ethernet cards, and/or any other types of interface devices that can send and receive information. Any suitable number of interfaces may be used to perform the described functions according to particular needs.

As discussed herein, controller 110 may be configured to evaluate supply chains by simulating how risks interact and overlap with components of the supply chains across time and space. Controller 110 may utilize processor 220 to evaluate risks to supply chains in this way. Processor 220 may include, for example, microprocessors, digital signal processors (DSPs), application specific integrated circuits (ASICs), field-programmable gate arrays (FPGAs), and/or equivalent discrete or integrated logic circuits. Two or more of processor 220 may be configured to work together to evaluate risks to supply chains over time and space.

Processor 220 may evaluate risks to supply chains according to instructions 232 stored on memory 230 of controller 110. Memory 230 may include a computer-readable storage medium or computer-readable storage device. In some examples, memory 230 may include one or more of a short-term memory or a long-term memory. Memory 230 may include, for example, random access memories (RAM), dynamic random-access memories (DRAM), static random-access memories (SRAM), magnetic hard discs, optical discs, floppy discs, flash memories, forms of electrically programmable memories (EPROM), electrically erasable and programmable memories (EEPROM), or the like. In some examples, processor 220 may analyze risks to supply chains according to instructions 232 of one or more applications (e.g., software applications) stored in memory 230 of controller 110.

In addition to instructions 232, in some examples gathered or predetermined data or techniques or the like as used by processor 220 to analyze risks to supply chains may be stored within memory 230. For example, memory 230 may include data from supply chain database 130 as received and/or gathered by controller 110, and/or memory 230 may include substantially all of supply chain database 130. Specifically, as depicted in FIG. 3, memory 230 may include component data 234, time data 236, location data 238, and risk data 240. Controller 110 may store some or all component data 234, time data 236, and location data 238 in a structure to maintain the interrelations as defined by supply chain as to how components 134 is to travel to locations 136 by points in time 138. For example, controller 110 may store component data 234, time data 236, and/or location data 238 in a two-dimensional spreadsheet, and/or in a three-dimensional spreadsheet, or in any other structure consistent with this disclosure that may store and define these relationships. As stored in memory 230, component data 234 may include specs on what component 134 must be and time data 236 may include threshold timeframes at which component 134 must be at locations 136 as defined by location data 238.

Further, as discussed above, memory 230 may include risk data 240. Risk data 240 may include current data on what known risks are, as well as any publicly available forecasts for risks. Risk data 240 may also include historical data that is related to risks (e.g., previous instances of events that were similar to a projected reality of risks).

Memory 230 may include preference and threshold data 244. For example, preference and threshold data 244 may include data such as risk tolerances of a user, a threshold that reflects this risk tolerances, inefficiency tolerances for options, and/or relations between risks and inefficiencies. Preference and threshold data 244 may also define criteria in which controller 110 may autonomously act in changing the supply change in response to a detected risk 132 that surpasses a threshold. For example, preference and threshold data 244 may detail that if a detected risk 132 is relatively severe (e.g., it will have a substantial impact upon a supply chain such that a business has more than a 50% chance of failing a contractual obligation related to this supply chain) and also that a change would include a reversion to a procedure that the supply chain regularly utilizes (e.g., a different season supplier), that controller 110 may autonomously execute the change to supply chain.

Memory 230 may further include machine learning techniques 242 that controller 110 may use to improve a process of identify and simulating risk events for a supply chain over time and space across iterations. For example, controller 110 may improve a process of calculating a likelihood of the risks impacting the supply chain. For another example, controller 110 may use machine learning techniques 242 to improve a process of selecting changes to the supply chain that mitigate the risk and also are acceptable to a user (and/or may be autonomously executed). For yet another example, controller 110 may use machine learning techniques 242 to improve a process of determining an amount of predicted inefficiency of an optional change to the supply chain.

Machine learning techniques 242 can comprise algorithms or models that are generated by performing supervised, unsupervised, or semi-supervised training on a dataset, and subsequently applying the generated algorithm or model to identify risks to a supply chain and then simulating these risks over space and time to determine if these risks surpass a threshold likelihood of negatively impacting the supply chain. Machine learning techniques 242 can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, the machine learning techniques 242 can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), partial least squares regression (PLSR), principal component analysis (PCA), principal component regression (PCR), Sammon mapping, t-distributed stochastic neighbor embedding (t-SNE), bootstrap aggregating, ensemble averaging, gradient boosted decision tree (GBRT), gradient boosting machine (GBM), inductive bias algorithms, Q-learning, state-action-reward-state-action (SARSA), temporal difference (TD) learning, apriori algorithms, equivalence class transformation (ECLAT) algorithms, Gaussian process regression, gene expression programming, group method of data handling (GMDH), inductive logic programming, instance-based learning, logistic model trees, information fuzzy networks (IFN), hidden Markov models, Gaussian naïve Bayes, multinomial naïve Bayes, averaged one-dependence estimators (AODE), Bayesian network (BN), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning techniques.

Memory 230 may include NLP techniques 246 that controller 110 may use to identify whether data stored in repositories 140 relates to risks 132 and/or may be used to simulate risks 132 over space and time. NLP techniques 246 can include, but are not limited to, semantic similarity, syntactic analysis, and ontological matching. For example, in some embodiments, processor 220 may be configured to parse comments from a news source or the like in repositories 140 to determine semantic features (e.g., word meanings, repeated words, keywords, etc.) and/or syntactic features (e.g., word structure, location of semantic features in headings, title, etc.) of an article regarding viral pandemics. Ontological matching could be used to map semantic and/or syntactic features to determine that this viral pandemic outbreak is in a part of a country through which the supply chain exists. Such concepts may also be used to identify previous similar outbreaks in this region and/or of a similar type of pandemic outbreak. In this way, using NLP techniques 246, controller 110 may, e.g., identify publicly available risk data that can be used to first identify a risk and then simulate these risks across space and time as they impact a supply chain.

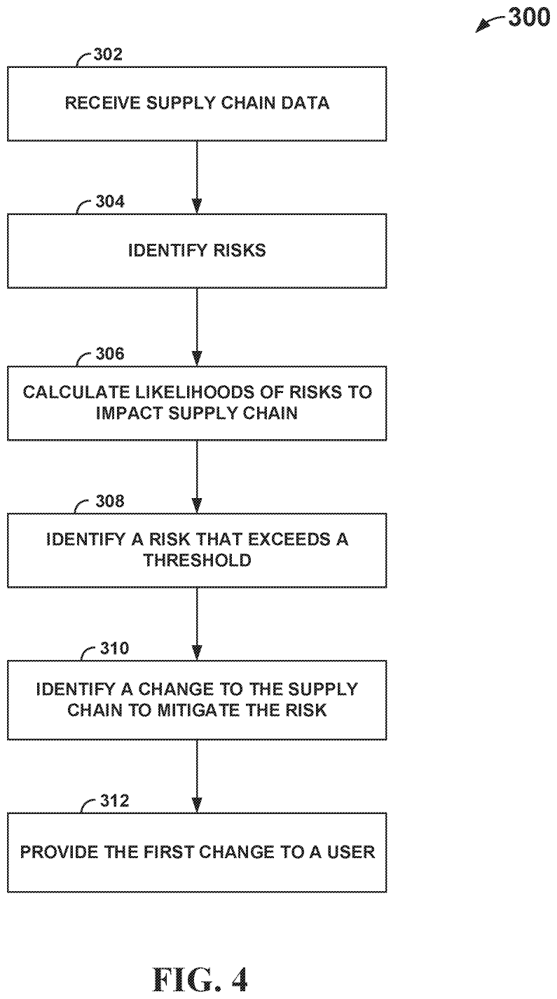

Using these components, controller 110 may identify risks to a supply chain and then simulate how these risks may impact the supply chain across time and space for a supply chain as discussed herein. For example, controller 110 may analyze risks for a supply chain according to flowchart 300 depicted in FIG. 4. Flowchart 300 of FIG. 4 is discussed with relation to FIG. 1 for purposes of illustration, though it is to be understood that other systems may be used to execute flowchart 300 of FIG. 4 in other examples. Further, in some examples controller 110 may evaluate risks differently than flowchart 300 of FIG. 4, or controller 110 may evaluate risks via a similar method with more or less steps in a different order, or the like.

Flowchart 300 begins with controller 110 receiving supply chain data (302). Controller 110 may receive this data from user device 120, and/or controller 110 may receive this data from supply chain database 130. Supply chain data may include data on a plurality of components 134 that are bought, transported, assembled, constructed, sold, or the like as part of supply chain. Supply chain data may also include determined one or more locations 136 for each of these components 134 at which and/or through which components 134 are bought, transported, assembled, constructed, sold, etc. Further, the supply chain data may include one or more points in time 138 associated with each component 134.

As discussed herein, points in time 138 may be relative to other components 134, such that, e.g., a supply chain defines that a first component 134 must be at a first location 136 at a first point in time 138 to ensure that a second component 134 will get to a second location 136 at a second point in time 138 (and/or that the first component 134 gets to the second location 136 at the second point in time 138). In this way, as stored within supply chain database 130 and as utilized by controller 110, each component 134, each location 136, and each point in time 136 may be interrelated.

Controller 110 may identify a plurality of risks 132 (304). For example, risks 132 may include geopolitical events, natural events such as wildfires or hurricanes, viral pandemics, or the like. Risks 132 may have the ability to negatively impact the supply chain. For example, risks 132 may reduce a likelihood that components 132 will move through locations 134 by requisite points in time 136, such that a supply chain has a chance of not delivering goods according to a predetermined timeline.

Controller 110 calculates a likelihood of each of these risks 132 impacting the supply chain (306). For example, controller 110 may calculate a likelihood that risks 132 may spread across time and/or space such that any component 134 is not able to get to a respective location 136 by a respective point in time 138, such that obligations of a supply chain are less likely to be met. For example, controller 110 may gather information of risks 132 from one or more repositories 140 to determine a current state of risks 132 and/or potential evolutions of risks 132. Controller 110 may further gather historical information on similar risks 132 from repositories 140. Using this gathered information on risks 132, controller 110 may simulate (e.g., using a neural network) how risks 132 spread across space and time.

In some examples, controller 110 may calculate the likelihood of risks 132 by simulating risks 132 and or components 134 as they move through locations 136 at times 138. Controller 110 may execute a predetermined number of simulations, and/or controller 110 may execute simulations until controller 110 can calculate likelihoods with at least a threshold confidence score. Controller 110 may simulate how risks 132 overlap with many components 134 across locations 136 and times 138, including how a delay to a first component 134 at a first location 136 at a first time 138 may impact other components 134 at other locations 136 at other times 138.

Controller 110 identifies one or more risks 132 that exceeds a threshold (308). Risks 132 exceeding a threshold may indicate that the respective risk 132 is relatively likely to occur, such that at least one component 134 has less than a threshold chance to get to a respective location 136 by a respective point in time 138. In response to identifying this risk 132, controller 110 identifies a first and second change to the supply chain to mitigate this risk (310). For example, controller 110 may determine to use a different supplier for component 134, to go to a different location 136, and/or to alter an order of events to change required respective points in time 138 at which component 134 must be at a respective location 136 to satisfy supply chain.

Once controller 110 identifies a first and second change to the supply chain, controller 110 may provide these changes to a user (312). Controller 110 may provide these changes to user device 120 of user, such as causing user device 120 to provide a graphical indication via graphical user interface 122 of user device 120. In some examples, controller 110 may further autonomously execute such changes to the supply chain. For example, controller 110 may autonomously instruct drivers to change to a different route, and/or tell a manufacturing assembly to keep assembling a first part of a product when component 134 needed for a second part of the product is likely to be delayed, or the like. Controller 110 may autonomously execute changes to the supply chain when controller 110 determines that a magnitude of respective risks 132 surpass a threshold, when controller 110 determines that an ease of an option is sufficiently low (e.g., when it is standard for an organization to use one of the first and/or second change in various circumstances), or the like.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
receiving data of a supply chain, wherein the data includes a plurality of components each moving through a plurality of locations at a plurality of points in time;
identifying a plurality of risks that relate to at least some of the plurality of locations;
calculating likelihoods of each of the plurality of risks impacting any of the plurality of components at any of the plurality of locations at any of the plurality of points in time by simulating the risk spreading across space and time;
identifying that a risk of the plurality of risks has more than a threshold likelihood of impacting at least one component of the plurality of components at a respective location of the plurality of locations at a respective point in time of the plurality of points in time; and
providing, to a user, a change to the supply chain that lowers a likelihood of the risk impacting the at least one component at the respective location at the respective point in time to below the threshold likelihood.

2. The computer-implemented method of claim 1, wherein the respective location is a first location and the respective point in time is a first point in time, further comprising identifying the change which includes causing the at least one component to move through an alternate location at the first point in time rather than the first location at the first point in time.

3. The computer-implemented method of claim 2, wherein the supply chain included a first supplier providing the at least one component via the first location, wherein the change includes a second supplier providing the at least one component via the alternate location.

4. The computer-implemented method of claim 2, further comprising providing the change to the user with a graphical indication via a user interface of a computing device of the user.

5. The computer-implemented method of claim 1, further comprising autonomously executing the change in response to determining that the change satisfied one or more criteria.

6. The computer-implemented method of claim 1, wherein the risk is a viral pandemic.

7. The computer-implemented method of claim 1, wherein a neural network identifies the risk.

8. A system comprising:
a processor; and
a memory in communication with the processor, the memory containing instructions that, when executed by the processor, cause the processor to:
receive data of a supply chain, wherein the data includes a plurality of components each moving through a plurality of locations at a plurality of points in time;
identify a plurality of risks that relate to at least some of the plurality of locations;
calculate likelihoods of each of the plurality of risks impacting any of the plurality of components at any of the plurality of locations at any of the plurality of points in time by simulating the risk spreading across space and time;
identify that a risk of the plurality of risks has more than a threshold likelihood of impacting at least one component of the plurality of components at a respective location of the plurality of locations at a respective point in time of the plurality of points in time; and
provide, to a user, a change to the supply chain that lowers a likelihood of the risk impacting the at least one component at the respective location at the respective point in time to below the threshold likelihood.

9. The system of claim 8, wherein the respective location is a first location and the respective point in time is a first point in time, further comprising identifying the change which includes causing the at least one component to move through an alternate location at the first point in time rather than the first location at the first point in time.

10. The system of claim 9, wherein the supply chain included a first supplier providing the at least one component via the first location, wherein the change includes a second supplier providing the at least one component via the alternate location.

11. The system of claim 9, the memory containing additional instructions that, when executed by the processor, cause the processor to provide the change to the user with a graphical indication via a user interface of a computing device of the user.

12. The system of claim 9, the memory containing additional instructions that, when executed by the processor, cause the processor to autonomously execute the change in response to determining that the change satisfied one or more criteria.

13. The system of claim 8, wherein the risk is a viral pandemic.

14. The system of claim 8, wherein a neural network identifies the risk.

15. A computer program product, the computer program product comprising a computer readable storage medium having program instructions embodied therewith, the program instructions executable by a computer to cause the computer to:
receive data of a supply chain, wherein the data includes a plurality of components each moving through a plurality of locations at a plurality of points in time;
identify a plurality of risks that relate to at least some of the plurality of locations;
calculate likelihoods of each of the plurality of risks impacting any of the plurality of components at any of the plurality of locations at any of the plurality of points in time by simulating the risk spreading across space and time;
identify that a risk of the plurality of risks has more than a threshold likelihood of impacting at least one component of the plurality of components at a respective location of the plurality of locations at a respective point in time of the plurality of points in time; and
provide, to a user, a change to the supply chain that lowers a likelihood of the risk impacting the at least one component at the respective location at the respective point in time to below the threshold likelihood.

16. The computer program product of claim 15, wherein the respective location is a first location and the respective point in time is a first point in time, further comprising identifying the change which includes causing the at least one component to move through an alternate location at the first point in time rather than the first location at the first point in time.

17. The computer program product of claim 16, wherein the supply chain included a first supplier providing the at least one component via the first location, wherein the change includes a second supplier providing the at least one component via the alternate location.

18. The computer program product of claim 16, the computer readable storage medium having additional program instructions that, when executed by the computer, cause the computer to provide the change to the user with a graphical indication via a user interface of a computing device of the user.

19. The computer program product of claim 16, the computer readable storage medium having additional program instructions that, when executed by the computer, cause the computer to autonomously execute the change in response to determining that the change satisfied one or more criteria.

20. The computer program product of claim 15, wherein the risk is a viral pandemic.
